(19) United States
(12) Patent Application Publication
(10) Pub. No.: US 2023/0003220 A1
(43) Pub. Date: Jan. 5, 2023

(54) MEDICAL FLUID PUMP HAVING AN ENERGY STORAGE COMPARTMENT

(71) Applicant: B. Braun Melsungen AG, Melsungen (DE)

(72) Inventors: Jan Schwarz, Melsungen (DE); Matthias Schwalm, Schwalmstadt (DE); Carsten Niemeier, Kassel (DE); Joachim Schuetz, Fulda (DE); Gerhard Schoeffel, Blaustein (DE); Daniel Wolf, Bibertal (DE)

(21) Appl. No.: 17/849,655

(22) Filed: Jun. 26, 2022

(30) Foreign Application Priority Data
Jul. 1, 2021 (DE) 20 2021 103 510.2

(51) Int. Cl. F04C 18/00; F01C 19/00

(52) U.S. Cl. CPC F04C 18/00; F01C 19/005

(57) ABSTRACT

A medical fluid pump with an upper housing shell, a lower housing shell and a front lid pivotably hinged to the lower housing shell. An energy storage compartment is formed on the lower housing shell which is provided and formed to receive an energy storage.

### CROSS-REFERENCE TO RELATED APPLICATION

This application claims priority under 35 U.S.C. § 119 to German Application No. 20 2021 103 510.2, filed Jul. 1, 2021, the content of which is incorporated by reference herein in its entirety.

### FIELD

The present disclosure relates to a medical fluid pump having an energy storage compartment.

### BACKGROUND

In medicine, fluid pumps, in particular syringe pumps and peristaltic pumps, are widely used to supply a patient with a defined dose of medication. It is essential that the supply of the patient with medication is ensured even in the event of a temporary disconnection of the fluid pump from a mains voltage. Such an interruption of the mains voltage can occur, for example, during transport of the patient or in the event of a power failure.

In fluid pumps in use today, an energy storage in the form of an accumulator is therefore located on the rear side of the fluid pump (on the side opposite the control panel). However, since an energy storage compartment intended for the energy storage takes up a relatively large installation space and the rear side of the fluid pump is used for various connections, the positioning of the connections on the rear side of the fluid pump is limited.

### SUMMARY

The object of the present disclosure is therefore to provide an energy storage in a medical fluid pump without restricting the configuration of a rear side of the fluid pump that is important for connections.

This object is solved by the medical fluid pump according to the disclosure with an energy storage compartment.

The medical fluid pump includes an upper housing shell, a lower housing shell and a front lid pivotably hinged to the lower housing shell. An energy storage compartment is formed on the lower housing shell and is provided and formed to receive an energy storage.

In other words, the fluid pump has a housing that includes an upper housing shell and a lower housing shell. A front lid is hinged to the lower housing shell, which preferably includes operating elements and/or a display device. The lower housing shell furthermore has an energy storage compartment. The energy storage compartment is provided and formed to receive a corresponding energy storage, in particular completely. In other words, the energy storage compartment is fully integrated into the lower housing shell of the fluid pump. The energy storage is preferably an accumulator or a battery. However, other forms of energy storage for electrical energy, such as a capacitor, are also conceivable.

Providing the energy storage compartment in the lower housing shell ensures that connections for power lines and/or data lines and/or substance lines can be freely positioned at the rear side of the fluid pump. In addition, by providing the energy storage compartment in the lower housing shell, the energy storage, which has a high weight, is mounted at the lowest possible position in the fluid pump, which ensures a favorable weight distribution and a safe standing of the fluid pump.

In a first aspect, the energy storage compartment comprises an opening at/in the lower housing shell.

In other words, the lower housing shell is provided with an opening that allows access to the energy storage. In particular, the opening enables insertion and removal of the energy storage compartment. Preferably, the opening is oriented towards the underside of the housing.

In a further aspect, the lower housing shell and the energy storage compartment are formed in one piece.

In other words, the energy storage compartment is formed as a part of the lower housing shell, in particular as a single piece of material. In this case, the lower housing shell with the energy storage compartment is preferably manufactured by injection molding.

In a further aspect, the medical fluid pump is provided with a cover that is provided and formed to close the opening of the energy storage compartment and, in particular, to be flush with the lower housing shell.

In other words, the cover is provided and formed to close the energy storage compartment and thus an interior space of the fluid pump from the environment. The cover is formed in such a way that when the cover closes the opening, a flat surface is created with an outer surface/skin of the lower housing shell.

On the one hand, the cover prevents the inserted energy storage from falling out of the energy storage compartment and, on the other hand, prevents foreign bodies from entering the interior of the fluid pump. The flat outer surface of the lower housing shell improves the handling and haptics of the fluid pump.

In a further aspect, the cover has a circumferential seal on its inner side that seals the cover against a sealing surface formed in the lower housing shell.

In other words, on the surface of the cover that faces inward in a state in which the cover closes the opening, a seal is provided and formed that completely surrounds the opening in the state in which the cover closes the opening. A sealing surface is provided and formed on the lower housing shell, which completely surrounds/encircles the opening, against which the cover seals in an assembled state via the seal.

The seal that completely surrounds the opening ensures that moisture cannot enter the fluid pump and damage electronic components in particular, such as the energy storage, even in a damp or wet operating environment.

In a further aspect, the cover is fixed with screws to the lower housing shell of the medical fluid pump, wherein cover caps are provided and formed to cover heads of the screws (screw heads) in an assembled state and in particular to be flush with the cover.

In other words, threaded holes are formed in the lower housing shell into which the screws are screwed and which fix the cover to the lower housing shell. Through holes for the screws are formed in the cover. On the outer surface of the cover, cylindrical depressions are preferably provided and formed around the through holes. These cylindrical depressions are formed to receive cover caps, preferably in a force-fit manner. In a state inserted into the depressions, the cover caps are flush with the outer surface of the cover. The cover caps are preferably made of an elastic material such as rubber. Preferably, the cover caps are provided and formed to be destructively removed.

The cover caps prevent dirt and moisture from entering the through holes/screw openings. In particular, the cover caps prevent moisture from entering the interior of the fluid pump through the through holes in the cover past the screw. Furthermore, the cover caps prevent unauthorized persons from opening the cover. The closed, flat surface also improves the handling and feel of the fluid pump.

In a further aspect, the energy storage compartment is provided with a notch that is provided and formed to receive an energy storage interconnecting cable.

In other words, the energy storage compartment includes a notch that allows the energy storage interconnecting cable connecting the energy storage to loads of the fluid pump and preferably to a mains voltage supply to be routed in a predetermined manner.

A separately formed notch for the energy storage interconnecting cable prevents the energy storage cable from being damaged or pulled out of a connector when the fluid pump is moved.

In a further aspect, the extension of the energy storage compartment from the lower housing shell toward the upper housing shell is greater than the extension of the energy storage compartment from the front lid toward a pump rear side opposite the front lid.

In other words, the extension of the energy storage compartment in the direction from a fluid pump bottom side towards a fluid pump top side is greater than the extension of the energy storage compartment in the direction from a fluid pump front side towards a fluid pump rear side. The fluid pump bottom side is preferably provided with feet. The fluid pump front side is preferably provided with a control panel.

In a further aspect, the energy storage compartment has a recess oriented in the direction of the front lid.

In other words, the energy storage compartment is provided with a material recess in a direction parallel to the front lid or normal to the fluid pump bottom side, respectively.

The recess allows easy removal of the energy storage by a technician. In addition, the recess provides better air circulation around the energy storage, which ensures that the energy storage does not heat up excessively during discharging and/or charging.

In another aspect, at least one buffer element is provided and formed on the cover on the side facing the energy storage compartment.

In other words, at least one elastic buffer element is attached to the inside of the cover. When the cover is mounted on the underside of the housing, the elastic buffer element is compressed between the cover and the energy storage. Preferably, the buffer element is provided and formed in the form of a foam bead.

The buffer element holds the energy storage in position and absorbs shocks and vibrations, which protects the energy storage from external force, for example if the fluid pump falls.

### BRIEF DESCRIPTION OF THE DRAWING FIGURES

FIG. 1 is a perspective view of a medical fluid pump according to the disclosure.

FIG. 2 is a perspective view of the medical fluid pump according to the disclosure with an opened cover of an energy storage compartment.

FIG. 3 is a detailed view of the medical fluid pump according to the disclosure with the energy storage removed.

FIG. 4 shows a detailed view of the medical fluid pump according to the disclosure.

FIG. 5 shows a top view of the energy storage compartment of the medical fluid pump according to the disclosure.

FIG. 6 shows an interior view of a lower housing shell of the medical fluid pump according to the disclosure.

FIG. 7 shows the cover of the energy storage compartment.

### DETAILED DESCRIPTION

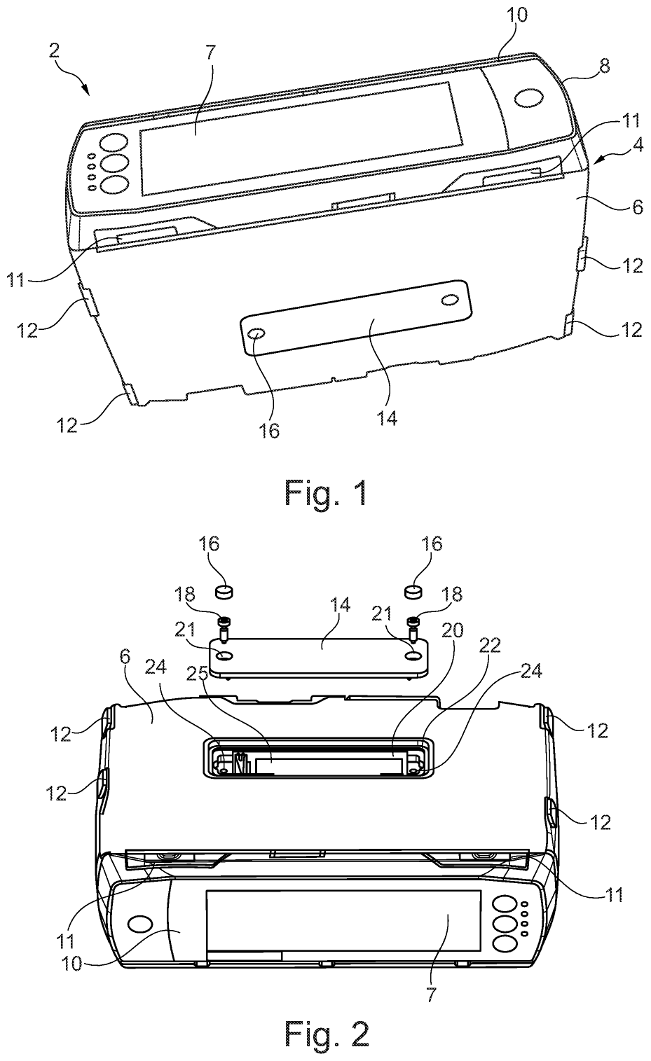

FIG. 1 shows a medical fluid pump 2 according to the disclosure with a housing 4, which includes a lower housing shell 6 and an upper housing shell 8. A front lid 10 is formed on a front side of the fluid pump 2, which is hinged to the lower housing shell 6 via hinges 11 in a foldable manner and which is provided and formed to contain an operating and display element 7 of the fluid pump 2. Feet 12 and a cover 14 of an energy storage compartment (20 in FIG. 2) are formed on an underside of the fluid pump 2 formed by the lower housing shell 6. The cover 14 is flush with the underside of the fluid pump 2. Cover caps 16 are formed on the cover 14.

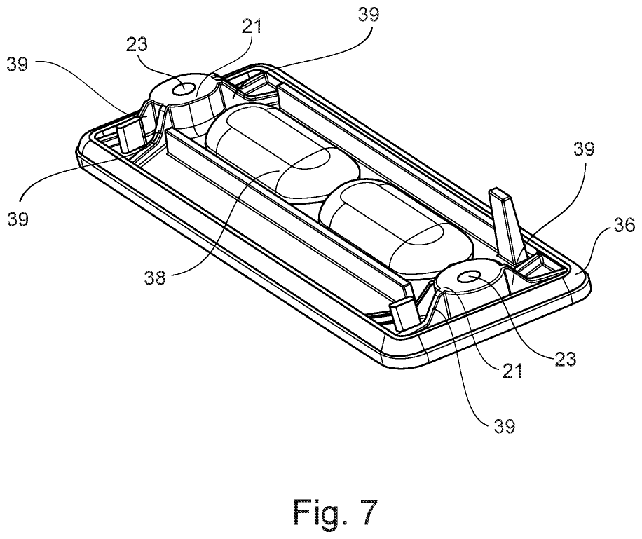

FIG. 2 shows the medical fluid pump 2 in a state in which the cover 14 of the energy storage compartment 20 is open. The cover 14 can be/is attached to the fluid pump 2 via screws 18 or can be/is attached to the fluid pump 2 via screws 18 such that an opening of the energy storage compartment 20 is closed. The cover 14 includes, on an outer surface (a surface facing away from the energy storage compartment 20 in an assembled state), cylindrical depressions 21 that are provided and formed to receive the cover caps 16. In this case, the cylindrical depressions 21 are located above through holes 23 of the cover 14, through which the screws 18 are screwed in order to fasten the cover 14 to the fluid pump 2. The cover caps 16 prevent moisture and contaminants from entering an interior of the fluid pump 2 through the through holes 23 (see FIG. 7). The energy storage compartment 20 includes a circumferential sealing rim/sealing surface 22 and threaded holes 24 into which the screws 18 can be screwed. An energy storage 25 is provided and formed to be inserted/insertable into the energy storage compartment 20.

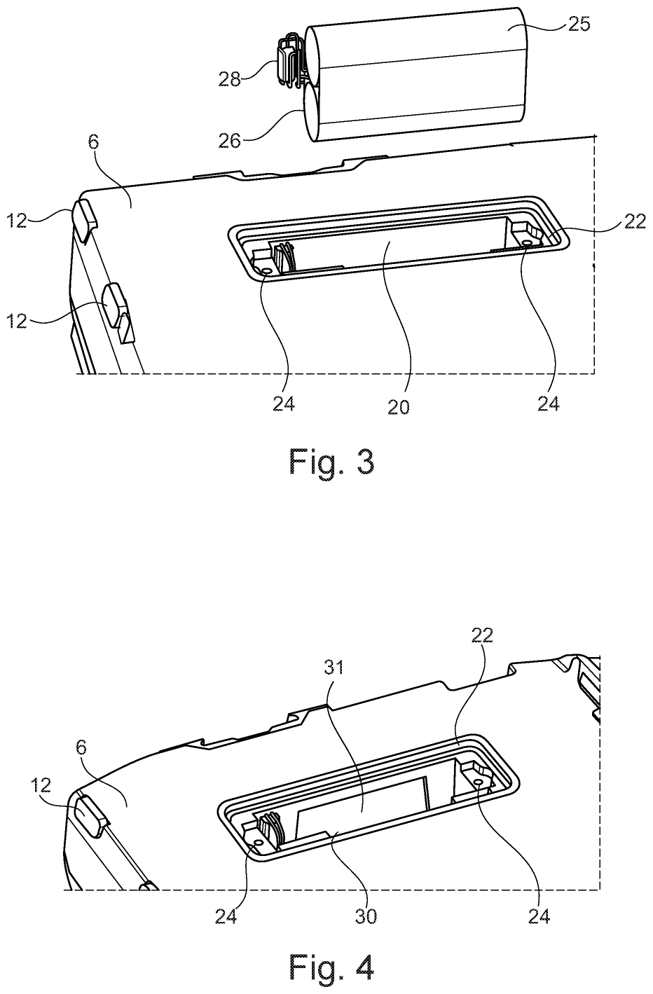

FIG. 3 shows a detailed view of the underside of the lower housing shell 6 with the opening of the energy storage compartment 20 and the removed energy storage 25. In a preferred embodiment, the energy storage 25 is an accumulator which, when the fluid pump 2 is connected to a mains voltage supply, is charged with mains voltage and, when the fluid pump 2 is disconnected from the mains voltage, supplies the fluid pump 2 with energy. Preferably, the energy storage is constructed of a plurality of energy storage elements 26 and is connected to the electronics of the fluid pump 2 via a supply connector 28. Preferably, the energy storage compartment 20 is arranged in the fluid pump 2 in such a way that the energy storage 25 in an installed state extends larger/wider in a direction normal to the underside of the fluid pump 2 than in the direction of the underside of the fluid pump 2.

FIG. 4 shows a detailed view of the underside of the lower housing shell 6 with the opening of the energy storage compartment 20. In the direction of the front lid 10, the energy storage compartment 20 has a recess 30. On the side facing away from the front lid 10, the energy storage compartment 20 has a cutout 31. The recess 30 and the cutout 31 ensure that air reaches the sides of the built-in energy storage compartment 25 and thus keeps the energy storage compartment 25 air-cooled. In addition, the recess 30 facilitates removal of the installed energy storage 25.

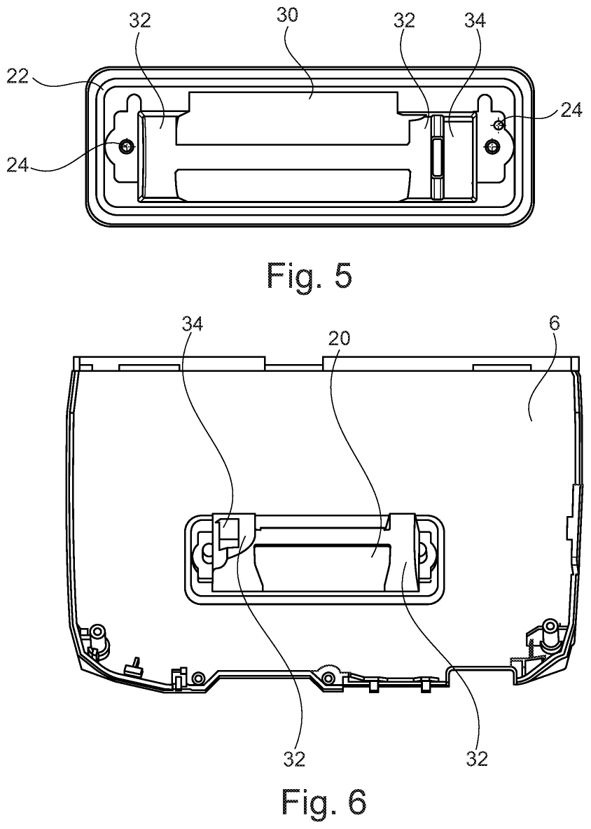

FIG. 5 shows a top view of the energy storage compartment 20 of the fluid pump 2. The energy storage compartment 20 has an energy-storage receiving portion 32, which is formed on the side of the energy storage compartment 20 facing away from the opening of the energy storage compartment 20. The energy-storage receiving portion 32 is provided and formed to receive the energy storage 25. Preferably, the shape of the energy-storage receiving portion 32 is adapted to the contour of the energy storage 25. The energy storage compartment 20 has a notch 34 that is provided and formed to receive the supply connector 28 that connects the electronics of the fluid pump 2 to the energy storage 25.

FIG. 6 shows an internal view of the lower housing shell 6 of the medical fluid pump 2 according to the disclosure. The energy storage compartment 20 is formed in one piece with the lower housing shell 6 and extends substantially normal to the lower housing shell 6.

FIG. 7 shows the cover 14 of the energy storage compartment 20, in particular an inner side of the cover 14 of the energy storage compartment 20. The inner side of the cover 14 is to be understood as the side that is oriented towards the interior space of the fluid pump 2 in a state in which the cover 14 is mounted on the fluid pump 2. On the inner side, a seal 36 is provided and formed circumferentially to seal the cover 14 against the sealing rim 22 of the energy storage compartment 20 in a mounted state. Furthermore, buffer elements 38 are provided and formed on the inner side. The buffer elements 38 are preferably foamed buffer elements 38 in the form of foam beads. When the cover 14 is screwed against the housing 4 of the fluid pump 2 via the screws 18 through the through holes 23, the seal 36 is pressed against the sealing rim 22 to form a seal. Furthermore, the buffer element 38 is pressed/clamped against the energy storage 25 in a mounted state and fixes the energy storage 25 between the cover 14 and the energy-storage receiving portion 32. Thus, the energy storage is prevented from slipping in the energy storage compartment 20 and the buffer element 38 protect the energy storage from mechanical stress. The depressions 21 are supported via struts 39 in the direction of the circumferential seal 36. In particular, the struts 39 are oriented in the direction of the corner sections of the circumferential seal 36. This ensures that the screws exert a uniform force, in particular in the corner sections of the circumferential seal 36, and further impedes the penetration of liquids.

1. A medical fluid pump comprising an upper housing shell, a lower housing shell and a front lid pivotably hinged to the lower housing shell, wherein an energy storage compartment is formed on the lower housing shell which is provided and formed to receive an energy storage.

2. The medical fluid pump according to claim 1, wherein the energy storage compartment comprises an opening at the lower housing shell.

3. The medical fluid pump according to claim 2, wherein the medical fluid pump comprises a cover that is provided and formed to close the opening.

4. The medical fluid pump according to claim 3, wherein the cover has a circumferential seal on its inner side that seals the cover against a sealing surface formed in the lower housing shell.

5. The medical fluid pump according to claim 3, wherein the cover is fixed with screws to the lower housing shell of the medical fluid pump, wherein cover caps are provided and formed to cover heads of the screws in an assembled state.

6. The medical fluid pump according to claim 3, wherein at least one buffer element is provided and formed on the cover on the side facing the energy storage compartment.

7. The medical fluid pump according to claim 1, wherein the lower housing shell and the energy storage compartment are formed in one piece.

8. The medical fluid pump according to claim 1, wherein the energy storage compartment is provided with a notch that is provided and formed to receive an energy storage connecting cable.

9. The medical fluid pump according to claim 1, wherein the extension of the energy storage compartment from the lower housing shell toward the upper housing shell is greater than the extension of the energy storage compartment from the front lid toward a pump rear side opposite the front lid.

10. The medical fluid pump according to claim 1, wherein the energy storage compartment has a recess oriented in the direction of the front lid.
